(19) United States
(12) Patent Application Publication
(10) Pub. No.: US 2025/0064703 A1
(43) Pub. Date: Feb. 27, 2025

(54) COMPOSITION COMPRISING SOLUBILIZED POLYPHENOL

(71) Applicant: L'OREAL, PARIS (FR)

(72) Inventors: Shinsuke OKUDA, Kawasaki-shi (JP); Toshifumi SHIROYA, Kawasaki-shi (JP); Hidehiko ASANUMA, Kawasaki-shi (JP)

(21) Appl. No.: 18/720,329

(22) PCT Filed: Dec. 6, 2022
(86) PCT No.: PCT/JP2022/045582

§ 371 Date: Jun. 14, 2024

(30) Foreign Application Priority Data
Dec. 20, 2021 (JP) 2021-205989
Jan. 19, 2022 (FR) 2200440

(51) Int. Cl. A61K 8/49; A61K 8/365; A61K 8/67; A61K 8/73; A61Q 19/08

(52) U.S. Cl. CPC A61K 8/498; A61K 8/365; A61K 8/676; A61K 8/736; A61Q 19/08

(57) ABSTRACT

A composition contains: (a) at least one polyphenol; (b) at least one first cationic polymer with a molecular weight of from 5,000 to 450,000, preferably from 10,000 to 350,000, and more preferably from 50,000 to 250, 000, selected from chitosans; (c) at least one organic compound selected from the group consisting of ascorbic acid, gluconolactone, gluconic acid, glycolic acid, and mixtures thereof; and (d) water. The pH of the composition is from 3.0 to 5.0. The (a) polyphenol can be solubilized in the composition.

### TECHNICAL FIELD

The present invention relates to a composition including a solubilized polyphenol, as well as a cosmetic process using the composition.

### BACKGROUND ART

Polyphenols are known to have anti-oxidative effects which could provide cosmetic effects such as anti-aging. Accordingly, cosmetic compositions comprising a polyphenol or polyphenols have been proposed. For example, WO 2017/167364 discloses a cosmetic composition including Hordeum vulgare seed extract including polyphenols.

### DISCLOSURE OF INVENTION

However, in general, it is difficult to solubilize a polyphenol into an aqueous composition. Therefore, utilization of the anti-oxidative effects of a polyphenol in an aqueous composition was limited.

Thus, an objective of the present invention is to provide an aqueous composition which comprises a polyphenol which is solubilized therein.

The above objective of the present invention can be achieved by a composition, comprising:
(a) at least one polyphenol;
(b) at least one first cationic polymer with a molecular weight of from 5,000 to 450,000, preferably from 10,000 to 350,000, and more preferably from 50,000 to 250,000, selected from chitosans;
(c) at least one organic compound selected from the group consisting of ascorbic acid, gluconolactone, gluconic acid, glycolic acid, and mixtures thereof; and
water (d)

wherein

the pH of the composition is from 3.0 to 5.0.

The (a) polyphenol may be selected from ellagic acid, punicalagins, and mixtures thereof.

The (a) polyphenol may be derived from plants, preferably Punica granatum.

The amount of the (a) polyphenol(s) in the composition according to the present invention may be from 0.001% to 10% by weight, preferably from 0.005% to 5% by weight, and more preferably from 0.01% to 1% by weight, relative to the total weight of the composition.

The (b) first cationic polymer may be selected from chitosans with an acetylation degree of more than 3% and less than 10%.

The amount of the (b) first cationic polymer(s) in the composition according to the present invention may be from 0.001% to 10% by weight, preferably from 0.005% to 5% by weight, and more preferably from 0.01% to 1% by weight, relative to the total weight of the composition.

The amount of the (c) organic compound(s) in the composition according to the present invention may be from 0.001% to 15% by weight, preferably from 0.005% to 10% by weight, and more preferably from 0.01% to 5% by weight, relative to the total weight of the composition.

The amount of the (d) water in the composition according to the present invention may be from 50% to 98% by weight, preferably from 60% to 95% by weight, and more preferably from 70% to 90% by weight, relative to the total weight of the composition.

The composition according to the present invention may further comprise (e) at least one second cationic polymer selected from polylysines.

The amount of the (e) second cationic polymer(s) in the composition according to the present invention may be from 0.001% to 10% by weight, preferably from 0.01% to 5% by weight, and more preferably from 0.1% to 3% by weight, relative to the total weight of the composition.

The composition according to the present invention may further comprise (f) at least one polyol.

It is preferable that the (f) polyol be dipropyleneglycol.

The amount of the (f) polyol(s) in the composition according to the present invention may be from 0.1% to 25% by weight, preferably from 0.5% to 20% by weight, and more preferably from 1% to 15% by weight, relative to the total weight of the composition.

The composition according to the present invention may be a cosmetic composition, preferably a transparent cosmetic composition, and more preferably a transparent skin cosmetic composition.

The present invention also relates to a cosmetic process for a keratin substance such as skin, comprising: applying to the keratin substance the composition according to the present invention.

### BEST MODE FOR CARRYING OUT THE INVENTION

After diligent research, the inventors have discovered that it is possible to provide an aqueous composition which comprises a polyphenol which is solubilized therein.

Thus, the composition according to the present invention comprises:
(a) at least one polyphenol;
(b) at least one first cationic polymer with a molecular weight of from 5,000 to 450,000, (b) preferably from 10,000 to 350,000, and more preferably from 50,000 to 250,000, selected from chitosans;
(c) at least one organic compound selected from the group consisting of ascorbic acid, gluconolactone, gluconic acid, glycolic acid, and mixtures thereof; and
water (d)

wherein

the pH of the composition is from 3.0 to 5.0.

The (a) polyphenol can be solubilized in the composition according to the present invention.

The solubility of the (a) polyphenol can be evaluated by the transparency of the composition according to the present invention. For example, if the composition according to the present invention is homogeneous and transparent, this means that the (a) polyphenol in the composition is well solubilized therein.

It is preferable that the solubility of the (a) polyphenol in the composition according to the present invention be maintained for a long period of time. Thus, it is preferable that the (a) polyphenol be solubilized in the composition according to the present invention for a long period of time. In other words, it is preferable that the transparency of the composition according to the present invention be maintained for a long period of time.

The composition according to the present invention may be transparent for one month or more.

The (a) polyphenol solubilized in the composition according to the present invention can exert good anti-oxidative effects which could contribute to cosmetic effects such as anti-aging.

Thus, the composition according to the present invention can provide good cosmetic effects based on the anti-oxidative effects due to the (a) polyphenol therein.

Hereinafter, the present invention will be explained in a more detailed manner.

### [Composition]

The composition according to the present invention comprises:
(a) at least one polyphenol;
(b) at least one first cationic polymer with a molecular weight of from 5,000 to 450,000, preferably from 10,000 to 350,000, and more preferably from 50,000 to 250,000, selected from chitosans;
(c) at least one organic compound selected from the group consisting of ascorbic acid, gluconolactone, gluconic acid, glycolic acid, and mixtures thereof; and
(d) water

wherein

the pH of the composition is from 3.0 to 5.0.

### (Polyphenol)

The composition according to the present invention comprises (a) at least one polyphenol. A single type of polyphenol may be used, or two or more different types of polyphenols may be used in combination.

The expression “polyphenol” is understood to mean a compound containing a plurality of phenolic hydroxyl groups. A phenolic hydroxyl group means a hydroxyl group bonded to an aromatic ring such as a benzene ring and a naphthalene ring. The phenolic hydroxyl group may be optionally etherified or esterified.

The (a) polyphenol may have poor solubility in water. Thus, the (a) polyphenol may have a water-solubility of less than 1% by weight, less than 0.1% by weight, or less than 0.01% by weight at room temperature (20° C. to 25° C., preferably 25° C.) without the (b) first cationic polymer, the (c) organic compound, and the (d) water, explained later.

The (a) polyphenol may be an active ingredient or active compound in cosmetics or dermatological products. The term an “active” compound used herein means a compound which has a cosmetic or dermatological active property, such as anti-oxidative, whitening, and anti-bacterial effects. It is preferable that the (a) polyphenol be chosen from those which have an anti-oxidizing activity.

The (a) polyphenol which can be used in the present invention may be flavonoids and non-flavonoids.

The flavonoids may be selected from the group consisting of chalcones, flavones, flavanones, flavanols, flavonols, dihydroflavonols, isoflavonoids, neoflavonoids, catechins, anthocyanidins, tannins, and derivatives thereof. In particular, the flavonoids may be tannins and derivatives thereof.

The tannins and their derivatives may be selected from the group consisting of ellagic acid, geraniin, catechins, procyanidin, profisetinidin, prodelphinidin, and derivatives thereof such as ellagitannin. Among these tannins, the present invention may, in particular, preferably use ellagic acid and ellagitannin, as the (a) polyphenol because ellagic acid has anti-oxidative effects.

Ellagic acid is represented by the following chemical formula. Ellagic acid can be obtained, for example, from Minasolve (product name: Minacare Elage).

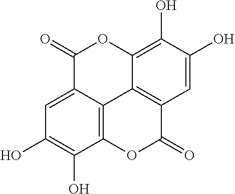 [SMILES: O=c1oc2c(O)c(O)cc3c(=O)oc4c(O)c(O)cc1c4c23]

Ellagitannin may be selected from the group consisting of castalagin, castalin, casuarictin, grandinin, roburin A, tellimagrandin II, terflavin B, vescalagin, punicalagin, punicalin, and mixtures thereof.

The non-flavonoids may be selected from the group consisting of lignans, aurones, stilbenoids, curcuminoids and other phenylpropanoids, and derivatives thereof, such as resorcinol derivatives. In particular, the non-flavonoids may be selected from stilbenoids and derivatives thereof and resorcinol derivatives.

The stilbenoids and derivatives may be selected from the group consisting of piceatannol, pinosylvin, resveratrol, and derivatives thereof. Among these stilbenoids, the present invention may preferably use resveratrol as the (a) polyphenol because resveratrol has whitening effects.

Resveratrol is represented by the following chemical formula. Resveratrol can be obtained, for example, from Symrise Corp.

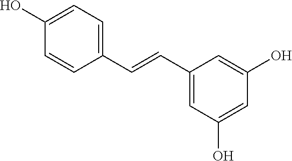 [SMILES: Oc1ccc(/C=C/c2cc(O)cc(O)c2)cc1]

The resorcinol derivative may preferably be 4-position substituted derivatives, such as 4-alkylresorcinols, more preferably phenyl ethyl resorcinol, 4-n-butylresorcinol and 4-(tetrahydro-2H-pyran-4-yl) benzene-1,3-diol, and in particular phenyl ethyl resorcinol because of its whitening effect. Phenyl ethyl resorcinol is also referred to as 4-(1-phenylethyl)-1,3-benzenediol and represented by the following chemical formula. Phenyl ethyl resorcinol can be obtained, for example, from Symrise Corp (product name: Symwhite 377®).

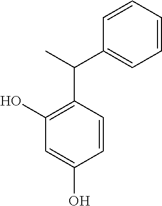 [SMILES: CC(c1ccccc1)c1ccc(O)cc1O]

As other examples of resorcinol derivatives, mention may be made of: 2-methylresorcinol, 5-methylresorcinol, 4-methylresorcinol, 4-ethylresorcinol, 2,5-dimethylresorcinol, 4,5-dimethylresorcinol, 2,4-dimethyl-1,3-benzenediol, 3,5-dihydroxybenzylamine, 5-methoxyresorcinol, 3,5-dihydroxybenzyl alcohol, 2-methoxyresorcinol, 4-methoxyresorcinol, 3,5-dihydroxytoluene monohydrate, 4-chlororesorcinol, 2-chlororesorcinol, 2′,4′-dihydroxyacetophenone, 3′,5′-dihydroxyacetophenone, 2,6-dihydroxy-4-methylbenzaldehyde, 4-propylresorcinol, 2,4-dihydroxy-1,3,5-trimethylbenzene, 3,5-dihydroxybenzamide, 2,6-dihydroxybenzamide, 2,4-dihydroxybenzamide, 2,4-dihydroxybenzoic acid, 2,6-dihydroxybenzoic acid, 3,5-dihydroxybenzoic acid, 2,6-dihydroxy-4-methylbenzyl alcohol, 3,5-dihydroxyanisole hydrate, 4-aminoresorcinol hydrochloride, 2-aminoresorcinol hydrochloride, 5-aminobenzene-1,3-diol hydrochloride, 2′,4′-dihydroxypropiophenone, 2′,4′-dihydroxy-3′-methylacetophenone, (2,4-dihydroxyphenyl) acetone, (3,5-dihydroxyphenyl) acetone, 2,6-dihydroxy-4′-methylacetophenone, 4-n-butylresorcinol, 2,4-diethyl-1,3-benzenediol, 3,5-dihydroxy-4-methylbenzoic acid, 2,6-dihydroxy-4-methylbenzoic acid, 2,4-dihydroxy-6-methylbenzoic acid, 3,5-dihydroxyphenylacetic acid, 2-ethyl-5-methoxybenzene-1,3-diol, 4-amino-3,5-dihydroxybenzoic acid, 3,5-dihydroxyacetophenone monohydrate, 3,5-dihydroxybenzylamine hydrochloride, 4,6-dichlororesorcinol, 2′,4′-dihydroxy-3′-methylpropiophenone, 1-(3-ethyl-2,6-dihydroxyphenyl)ethan-1-one, 2′,6′-dihydroxy-4′-methoxyacetophenone, 1-(2,6-dihydroxy-3-methoxyphenyl)ethan-1-one, 3 (2,4-dihydroxyphenylpropionic acid, and 2,4-dihydroxy-3,6-dimethylbenzoic acid.

The salts of the (a) polyphenol include conventional non-toxic salts of said compounds, such as those formed from an acid or from a base.

Salts of the (a) polyphenol include the following:

a) salts obtained by addition of the compound with a mineral acid, selected more particularly from hydrochloric, boric, hydrobromic, hydroic, sulphuric, nitric, carbonic, phosphoric and tetrafluoroboric acids; or

b) salts obtained by addition of the compound with an organic acid, more particularly selected from acetic, propionic, succinic, fumaric, lactic, glycolic, citric, gluconic, salicylic, tartaric, terephthalic, methylsulphonic, ethylsulphonic, benzene sulphonic, toluene sulphonic and triflic acids.

Also included are the salts obtained by addition of the (a) polyphenol with a mineral base, such as aqueous sodium hydroxide and potassium hydroxide, calcium hydroxide, ammonium hydroxide, magnesium hydroxide, lithium hydroxide, and sodium, potassium or calcium carbonates or hydrogencarbonates, for example; or with an organic base such as a primary, secondary or tertiary alkylamine, for example triethylamine or butylamine.

The salts of the (a) polyphenol may advantageously be selected from alkali metal salts or alkaline earth metal salts such as sodium, potassium, calcium and magnesium salts; and ammonium salts.

It is preferable that the (a) polyphenol be selected from ellagic acid, ellagitannin such as punicalagins and punicalin, and mixtures thereof.

Punicalagins mean a mixture of two diastereoisomeres, punicalagin A and B. They belong to the polyphenols family, and especially are complex ellagitannins formed of glucose linked to ellagic acid and gallagic acid of molar mass of about 1084 gmol-1 formed by the isomers of 2,3-(S)-hexahydroxydiphenoyl-4,6-(S,S)-gallagyl-D-glucose of the following chemical formula.

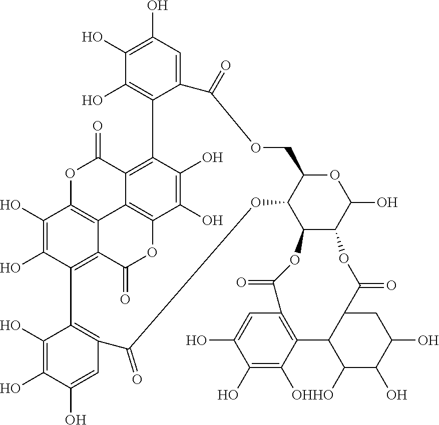 [SMILES: O=C1OC[C@H]2OC(O)[C@@H]3OC(=O)C4CC(O)C(O)C(O)C4c4c(cc(O)c(O)c4O)C(=O)O[C@H]3[C@@H]2OC(=O)c2cc(O)c(O)c(O)c2-c2c(O)c(O)c3oc(=O)c4c(c(O)c(O)c5oc(=O)c2c3c54)-c2c1cc(O)c(O)c2O]

The term “punicalins” means a mixture of two diastereoisomeres, punicalin A and B. They belong to the polyphenols family, and especially are ellagitannins named 4,6-(S,S)-Gallagyl-D-glucose of the following chemical formula.

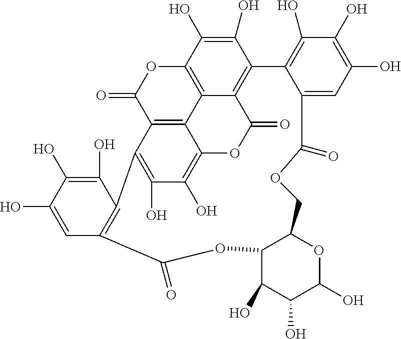 [SMILES: O=C1OC[C@H]2OC(O)[C@H](O)[C@@H](O)[C@@H]2OC(=O)c2cc(O)c(O)c(O)c2-c2c(O)c(O)c3oc(=O)c4c(c(O)c(O)c5oc(=O)c2c3c54)-c2c1cc(O)c(O)c2O]

It may be more preferable that the (a) polyphenol be selected from ellagic acid, punicalagins, and mixtures thereof.

It is preferable that the (a) polyphenol be derived from plants, and more preferably Punica granatum.

Punica granatum is a fruit-bearing deciduous shrub or small tree in the family Lythraceae that grows between five and 10 m (16-30 feet) in height. On multiple, spiny branches, the deciduous leaves are opposite or in whorls of five or six, short-stemmed, oblong-lanceolate, leathery, and 1-10 cm (0.4-4 in) long. Showy red, white, or variegated flowers are found on the branch tips, singly or in clusters of up to five flowers. Nearly round, but crowned at the base by the prominent calyx, the pomegranate fruit has a tough, leathery skin or rind, and is basically yellow overlaid with light or deep pink or rich red. The interior is separated by membranous walls and white spongy tissue into compartments packed with transparent sacs filled with tart, flavorful, fleshy, juicy pulp (the aril). In each sac, there is one white or red, angular, soft or hard seed. The arils represent about 52% of the weight of the whole fruit.

For the purposes of the present invention, the fruit and in particular the pericarp of Punica granatum is more preferred.

In a preferred embodiment of the present invention, the fruit of Punica granatum is collected from the India state of Jammu and Kashmir around the Ramban district (kanga village) during the period of September to December, more particularly at the latitude and longitude around 32° N, 74° E.

Punica granatum Extract:

According to one embodiment of the present invention, an extract of Punica granatum (Punica granatum extract) may be used as the (a) polyphenol.

Preferably, the above Punica granatum extract may comprise: from 10% to 15% by weight of the total weight of the dry extract of punicalagins, preferably from 10.5% to 15%, more preferably from 10.8% to 14.8%, and even more preferably from 11.7% to 14.6%; and from 10% to 15% by weight of the total weight of the dry extract of ellagic acid, preferably from 10.5% to 14.5%, more preferably from 10.8% to 14.8%, and even more preferably from 11.2% to 14.5%.

The Punica granatum extract may further comprise 1% to 5% by weight of the total weight of the dry extract of punicalins, preferably 2% to 4.5%, and even more preferably 3% to 4%.

Advantageously, the weight ratio [ellagic acid/punicalagins] may range from 0.6 to 1.6, more preferably from 0.7 to 1.3, and even more preferably from 0.7 to 1.

In a first preferred embodiment, the Punica granatum extract may comprise: from 11.7% to 14.6% by weight of the total weight of the dry extract of punicalagins, and from 11.2% to 14.5% by weight of the total weight of the dry extract of ellagic acid, wherein the weight ratio [ellagic acid/punicalagins] ranges from 0.6 to 1.6, more preferably from 0.7 to 1.3, and even more preferably from 0.7 to 1.

In a second preferred embodiment, the Punica granatum extract may comprise: from 11.7% to 14.6% by weight of the total weight of the dry extract of punicalagins, and from 11.2% to 14.5% by weight of the total weight of the dry extract of ellagic acid, and iii. from 3% to 4% by weight of the total weight of the dry extract of punicalins, wherein the weight ratio [ellagic acid/punicalagins] ranges from 0.6 to 1.6, more preferably from 0.7 to 1.3, and even more preferably from 0.7 to 1.

The Punica granatum extract may be either in the form of an ethanolic extract or a dry extract.

The term “dry extract” means an extract which comprises no more than 7% by weight of solvents relative to the total weight of the dry extract, preferably no more than 5%, and more preferably no more than 3% by weight of solvents relative to the total weight of the dry extract. In a preferred embodiment, the dry extract comprises no more than 1% by weight of solvents relative to the total weight of the dry extract. In another preferred embodiment, the dry extract does not contain any solvent (i.e. 0%).

The term “solvent” means aqueous, organic solvents and their mixtures.

As aqueous solvents, a mention may be made of water. As organic solvents, mention may be made of an alcoholic solvent, especially monoalcohol in C2-C5 such as those chosen from ethanol, propanol, isopropanol, butanol and pentanol, and their mixtures.

Method of Preparation of Punica granatum Extract:

The Punica granatum extract may be prepared by the following steps:
(i) providing a Punica granatum pericarp;
(ii) extracting from the said pericarp, at least punicalagins and ellagic acid, with ethanol of at least 99.5% at a temperature ranging from 37°° C. to 45° C. for 2 to 5 hours;
(iii) filtering and repeating step (ii) at least two more times; and
(iv) optionally drying the filtrate obtained at the end of step (iii) at a temperature ranging from 35° C. to 40° C. under vacuum.

Advantageously, ethanol in step (ii) has a moisture level less than 0.5%, more preferably less than 0.2%.

Preferably, the Punica granatum pericarp provided at step (i) is a dried pericarp in the form of a powder which especially comprises from 1% to 5% by weight of the total weight of the dried pericarp powder of punicalagins and from 1% to 5% by weight of the total weight of the dried pericarp powder of ellagic acid, more preferably from 2% to 3% by weight of the total weight of the dried pericarp powder of punicalagins and from 1% to 4% by weight of the total weight of the dried pericarp powder of ellagic acid.

In a preferred embodiment, the weight ratio [pericarp/absolute ethanol] is 1:5.

Advantageously, the temperature of the extracting step (ii) ranges from 40° C. to 43° C., and more preferably is 42° C.

Step (iii) of filtering is performed according to a conventional method of filtration well known by the skilled person in the art, for example by a Buchner funnel.

Preferably, the temperature of the drying step (iv) ranges from 37° C. to 39° C., and more preferably is 38° C.

In a particular embodiment, the method of preparation of the Punica granatum extract may comprise the following steps of:
(v) providing a Punica granatum pericarp;
(vi) extracting from the said pericarp, at least punicalagins and ellagic acid, with ethanol of at least 99.5% at a temperature ranging from 37° C. to 45° C. for 2 to 5 hours;
(vii) filtering and repeating step (ii) at least two more times; and
(viii) optionally drying the filtrate obtained at the end of step (iii) at a temperature ranging from 35° C. to 40° C. under vacuum.

The amount of the (a) polyphenol(s) in the composition according to the present invention may be 0.001% by weight or more, preferably 0.005% by weight or more, more preferably 0.01% by weight or more, and even more preferably 0.05% by weight, relative to the total weight of the composition.

The amount of the (a) polyphenol(s) in the composition according to the present invention may be 10% by weight or less, preferably 5% by weight or less, more preferably 1% by weight or less, and even more preferably 0.5% by weight or less, relative to the total weight of the composition.

The amount of the (a) polyphenol(s) in the composition according to the present invention may be from 0.001% to 10% by weight, preferably from 0.005% to 5% by weight, more preferably from 0.01% to 1% by weight, and even more preferably from 0.05% to 0.5% by weight or less, relative to the total weight of the composition.

### (First Cationic Polymer)

The composition according to the present invention comprises (b) at least one first cationic polymer. A single type of first cationic polymer may be used, or two or more different types of first cationic polymers may be used in combination.

The cationic polymer may have a positive charge density. The charge density of the (b) first cationic polymer may be from 0.01 meq/g to 20 meq/g, preferably from 0.05 meq/g to 15 meq/g, and more preferably from 0.1 meq/g to 10 meq/g.

The molecular weight of the (b) first cationic polymer is from 5,000 to 450,000, preferably from 10,000 to 350,000, and more preferably from 50,000 to 250,000.

Unless otherwise defined in the descriptions, “molecular weight” means number average molecular weight.

The (b) first cationic polymer is selected from chitosans. Chitosans comprise chitosan, salts of chitosan, and glycosides of chitosan.

Chitosan is well known. Chitosan can be a linear polysaccharide composed of randomly distributed β-(1→4)-linked D-glucosamine (deacetylated unit) and N-acetyl-D-glucosamine (acetylated unit).

The actylation degree of chitosan may be more than 3% to less than 10%, preferably from 3% to 8%, and more preferably from 3% to 5%.

Chitosan can be prepared by, for example, treating the chitin shells of shrimp and other crustaceans with an alkaline substance, such as sodium hydroxide. However, it is preferable that chitosan be derived from fungus such as Aspergillus Niger because the chitosan derived from fungus could be free from any allergen. Thus, it is preferable that the (b) first cationic polymer be derived from fungus. The amount of the (b) first cationic polymer(s) in the composition according to the present invention may be 0.001% by weight or more, preferably 0.005% by weight or more, and more preferably 0.01% by weight or more, relative to the total weight of the composition. The amount of the (b) first cationic polymer(s) in the composition according to the present invention may be 10% by weight or less, preferably 5% by weight or less, and more preferably 1% by weight or less, relative to the total weight of the composition.

The amount of the (b) first cationic polymer(s) in the composition according to the present invention may be from 0.001% to 10% by weight, preferably from 0.005% to 5% by weight, and more preferably from 0.01% to 1% by weight, relative to the total weight of the composition.

### (Organic Compound)

The composition according to the present invention comprises (c) at least one (specific) organic compound. Two or more specific organic compounds may be used in combination. Thus, a specific organic compound or a combination of specific organic compounds may be used.

The (c) organic compound is selected from the group consisting of ascorbic acid, gluconolactone, gluconic acid, glycolic acid, and mixtures thereof.

The (c) organic compound may be in the form of a salt. The term “salt” here means a salt formed by addition of suitable base(s) to the (c) organic compound, which may be obtained from a reaction with the (c) organic compound the base(s) according to methods known to those skilled in the art. As the salt, mention may be made of metal salts, for example salts with alkaline metal such as Na and K, and salts with alkaline earth metal such as Mg and Ca, and ammonium salts.

The amount of the (c) organic compound(s) in the composition according to the present invention may be 0.001% by weight or more, preferably 0.005% by weight or more, and more preferably 0.01% by weight or more, relative to the total weight of the composition.

The amount of the (c) organic compound(s) in the composition according to the present invention may be 15% by weight or less, preferably 10% by weight or less, and more preferably 5% by weight or less, relative to the total weight of the composition.

The amount of the (c) organic compound(s) in the composition according to the present invention may be from 0.001% to 15% by weight, preferably from 0.005% to 10% by weight, and more preferably from 0.01% to 5% by weight, relative to the total weight of the composition.

### (Water)

The composition according to the present invention comprises (d) water.

The amount of the (d) water in the composition according to the present invention may be 50% by weight or more, preferably 60% by weight or more, and more preferably 70% by weight or more, relative to the total weight of the composition.

The amount of the (d) water in the composition according to the present invention may be 98% by weight or less, preferably 95% by weight or less, and more preferably 90% by weight or less, relative to the total weight of the composition.

The amount of the (d) water in the composition according to the present invention may be from 50% to 98% by weight, preferably from 60% to 95% by weight, and more preferably from 70% to 90% by weight, relative to the total weight of the composition.

### (Second Cationic Polymer)

The composition according to the present invention may further comprise (e) at least one second cationic polymer. A single type of second cationic polymer may be used, or two or more different types of second cationic polymers may be used in combination.

The cationic polymer may have a positive charge density. The charge density of the (e) second cationic polymer may be from 0.01 meq/g to 20 meq/g, preferably from 0.05 meq/g to15 meq/g, and more preferably from 0.1 meq/g to 10 meq/g.

The molecular weight of the (e) second cationic polymer may be from 1,000 to 100,000, preferably from 1,500 to 50,000, and more preferably from 2,000 to 10,000.

Unless otherwise defined in the descriptions, “molecular weight” means number average molecular weight.

The (e) second cationic polymer is selected from polylysines. Polylysines comprise polylysine, and salts of polylysine.

Polylysine is well known. Polylysine can be a natural homopolymer of L-lysine that can be produced by bacterial fermentation. For example, polylysine can be ε-Poly-L-lysine, typically used as a natural preservative in food products.

Polylysine is a polyelectrolyte which is soluble in polar solvents such as water, propylene glycol and glycerol. Polylysine is commercially available in various forms, such as poly D-lysine and poly L-lysine.

The amount of the (e) second cationic polymer(s) in the composition according to the present invention may be 0.001% by weight or more, preferably 0.01% by weight or more, and more preferably 0.1% by weight or more, relative to the total weight of the composition.

The amount of the (e) second cationic polymer(s) in the composition according to the present invention may be 10% by weight or less, preferably 5% by weight or less, and more preferably 3% by weight or less, relative to the total weight of the composition.

The amount of the (e) second cationic polymer(s) in the composition according to the present invention may be from 0.001% to 10% by weight, preferably from 0.01% to 5% by weight, and more preferably from 0.1% to 3% by weight, relative to the total weight of the composition.

### (Polyol)

The composition according to the present invention may further comprise (f) at least one polyol. A single type of polyol may be used, but two or more different types of polyol may be used in combination.

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acyl group or a carbonyl group.

The polyol may be a C₂-C₁₂ polyol, preferably a C₂-C₉ polyol, comprising at least 2 hydroxy groups, and preferably 2 to 5 hydroxy groups.

The polyol may be a natural or synthetic polyol. The polyol may have a linear, branched or cyclic molecular structure.

The polyol may be selected from glycerins and derivatives thereof, and glycols and derivatives thereof. The polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, 1,3-propanediol, 1,5-pentanediol, polyethyleneglycol (5 to 50 ethyleneoxide groups), and sugars such as sorbitol.

It is preferable to use dipropyleneglycol as the (f) polyol.

The amount of the (f) polyol(s) in the composition according to the present invention may be 0.1% by weight or more, preferably 0.5% by weight or more, and more preferably 1% by weight or more, relative to the total weight of the composition.

The amount of the (f) polyol(s) in the composition according to the present invention may be 25% by weight or less, preferably 20% by weight or less, and more preferably 15% by weight or less, relative to the total weight of the composition.

The amount of the (f) polyol(s) in the composition according to the present invention may be from 0.1% to 25% by weight, and preferably from 0.5% to 20% by weight, such as from 1% to 15% by weight, relative to the total weight of the composition.

(pH)

The pH of the composition according to the present invention is from 3.0 to 5.0, preferably from 3.1 to 4.9, more preferably from 3.2 to 4.8, even more preferably from 3.3 to 4.7, and even still more preferably from 3.4 to 4.6, and further more preferably from 3.5 to 4.5.

At a pH of from 3.0 to 5.0, the (a) polyphenol can be solubilized well.

The pH of the composition according to the present invention may be adjusted by adding at least one alkaline agent and/or at least one acid, other than the (c) organic compound.

The pH of the composition according to the present invention may also be adjusted by adding at least one buffering agent.

### Alkaline Agent:

The composition according to the present invention may comprise at least one alkaline agent. Two or more alkaline agents may be used in combination. Thus, a single type of alkaline agent or a combination of different types of alkaline agents may be used.

The alkaline agent may be an inorganic alkaline agent. It is preferable that the inorganic alkaline agent be selected from the group consisting of ammonia; alkaline metal hydroxides; alkaline earth metal hydroxides; alkaline metal phosphates and monohydrogenophosphates such as sodium phosphate or sodium monohydrogen phosphate.

As examples of the inorganic alkaline metal hydroxides, mention may be made of sodium hydroxide and potassium hydroxide. As examples of the alkaline earth metal hydroxides, mention may be made of calcium hydroxide and magnesium hydroxide. As an inorganic alkaline agent, sodium hydroxide is preferable.

The alkaline agent may be an organic alkaline agent. It is preferable that the organic alkaline agent be selected from the group consisting of monoamines and derivatives thereof; diamines and derivatives thereof; polyamines and derivatives thereof; basic amino acids and derivatives thereof; oligomers of basic amino acids and derivatives thereof; polymers of basic amino acids and derivatives thereof; urea and derivatives thereof; and guanidine and derivatives thereof.

As examples of the organic alkaline agents, mention may be made of alkanolamines such as mono-, di-and tri-ethanolamine, and isopropanolamine; urea, guanidine and their derivatives; basic amino acids such as lysine, ornithine or arginine; and diamines such as those described in the structure below:

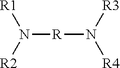

wherein R denotes an alkylene such as propylene optionally substituted by a hydroxyl or a C₁-C₄ alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof. Arginine, urea and monoethanolamine are preferable.

The alkaline agent(s) may be used in a total amount of from 0.01% to 15% by weight, preferably from 0.05% to 10% by weight, more preferably from 0.1% to 5% by weight, relative to the total weight of the composition, depending on their solubility.

### Acid:

The composition according to the present invention may comprise at least one acid. Two or more acids may be used in combination. Thus, a single type of acid or a combination of different types of acids may be used.

As the acid, mention may be made of any inorganic or organic acids, preferably inorganic acids, which are commonly used in cosmetic products.

A monovalent or polyvalent acid may be used.

The acid(s) may be used in a total amount of from 0.01% to 15% by weight, preferably from 0.05% to 10% by weight, and more preferably from 0.1% to 5% by weight, relative to the total weight of the composition, depending on their solubility.

### Buffering Agent:

The composition according to the present invention may comprise at least one buffering agent. Two or more buffering agents may be used in combination. Thus, a single type of buffering agent or a combination of different types of buffering agents may be used.

As the buffering agent, mention may be made of an acetate buffer (for example, acetic acid+sodium acetate), a phosphate buffer (for example, sodium dihydrogen phosphate+di-sodium hydrogen phosphate), a citrate buffer (for example, citric acid+sodium citrate), a borate buffer (for example, boric acid+sodium borate), a tartrate buffer (for example, tartaric acid+sodium tartrate dihydrate), Tris buffer (for example, tris (hydroxymethyl) aminomethane), and a Hepes buffer (4-(2-hydroxyethyl)-1-piperazineethanesulfonic acid).

### (Optional Ingredients)

The composition according to the present invention may comprise, in addition to the aforementioned ingredients, ingredients typically employed in cosmetics, specifically, surfactants/emulsifiers, hydrophilic or lipophilic thickeners, derived from, for example, synthetic polymers other than the (b) first cationic polymer and the (e) second cationic polymer; volatile or non-volatile organic solvents other than the (f) polyol; anionic polymers; amphoteric polymers; nonionic polymers; silicones and silicone derivatives; natural extracts derived from animals or vegetables other than those including the (a) polyphenol; waxes; and the like, within a range which does not impair the effects of the present invention.

The composition according to the present invention may comprise the above optional ingredient(s) in an amount of from 0.01% to 30% by weight, preferably from 0.05% to 20% by weight, and more preferably from 0.1% to 10% by weight, relative to the total weight of the composition.

The amount of the surfactant(s)/emulsifier(s) in the composition according to the present invention may be 1% by weight or less, preferably 0.1% by weight or less, and more preferably 0.01% by weight or less, relative to the total weight of the composition. It is particularly preferable that the composition according to the present invention includes no surfactant/emulsifier.

### [Preparation]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

It is preferable that the ingredient (a) is mixed with the ingredient (f) to obtain a mixture of the ingredients (a) and (f), and the mixture of the ingredients (a) and (f) is added to a mixture of the ingredients (b) to (d), and optionally the ingredient (e), which has already been prepared separately, to prepare the composition according to the present invention.

### [Cosmetic Composition]

The composition according to the present invention may be intended to be used as a cosmetic composition. Thus, the cosmetic composition according to the present invention may be intended for application onto a keratin substance. Keratin substance here means a material containing keratin as a main constituent element, and examples thereof include the skin, scalp, nails, lips, hair, and the like. Thus, it is preferable that the cosmetic composition according to the present invention be used for a cosmetic process for the keratin substance, in particular skin.

Thus, the cosmetic composition according to the present invention may be a skin cosmetic composition, preferably a skin care composition or a skin makeup composition, and more preferably a skin care composition.

The composition according to the present invention can have a transparent appearance.

Thus, the composition according to the present invention may be a cosmetic composition, preferably a transparent cosmetic composition, and more preferably a transparent skin cosmetic composition.

The transparency may be measured by measuring the turbidity (for example, turbidity can be measured with a 2100Q (marketed by HACH) having a round cell (25 mm in diameter and 60 mm height) and a tungsten filament lamp which can emit visible light (between 400 and 800 nm, preferably from 400 to 500 nm). The measurement can be performed on the undiluted composition. The blank may be determined with distilled water.

The composition according to the present invention may preferably have a turbidity of less than 200 NTU, preferably less than 150 NTU, more preferably less than 100 NTU, and even more preferably less than 50 NTU.

### [Cosmetic Process and Use]

The present invention also relates to:

a cosmetic process for a keratin substance such as skin, comprising: applying to the keratin substance the composition according to the present invention.

The cosmetic process here means a non-therapeutic cosmetic method for caring for and/or making up the surface of a keratin substance such as skin.

The present invention also relates to a use of a composition, comprising:
(b) at least one first cationic polymer with a molecular weight of from 5,000 to 450,000, preferably from 10,000 to 350,000, and more preferably from 50,000 to 250,000, selected from chitosans;
(c) at least one organic compound selected from the group consisting of ascorbic acid, gluconolactone, gluconic acid, glycolic acid, and mixtures thereof; and
(d) water

wherein

the pH of the composition is from 3.0 to 5.0,

in order to solubilize (a) at least one polyphenol in the composition.

The above explanations regarding the (a) polyphenol, the (b) first cationic polymer, the (c) organic compound, and the (d) for the composition according to the present invention can apply to those in the use according to the present invention.

The above use according to the present invention can make the composition transparent, preferably for 1 month or more.

### EXAMPLES

The present invention will be described in a more detailed manner by way of examples. However, they should not be construed as limiting the scope of the present invention.

### Examples 1-4 and Comparative Examples 1-7

### [Preparation]

Compositions according to Examples 1-4 and Comparative Examples 1-7 were prepared by mixing the ingredients shown in Tables 1 and 2. The amounts of each ingredient used in the above preparations are shown in Tables 1 and 2. The numerical values for the amounts of the ingredients shown in Tables 1 and 2 are all based on “% by weight” as raw materials.

TABLE 1

| | Ex. 1 | Ex. 2 | Ex. 3 | Ex. 4 |
|---|---|---|---|---|
| Water | qsp 100 | qsp 100 | qsp 100 | qsp 100 |
| Chitosan | 0.2 | 0.2 | 0.2 | 0.2 |
| (MW: 100,000) | | | | |
| (Acetylation Degree: 4) | | | | |
| Ascorbic Acid | 0.4 | — | — | 0.4 |
| Gluconolactone | — | 0.2 | — | — |
| Glycolic Acid | — | — | 0.1 | — |
| Dipropylene Glycol | 9.5 | 9.5 | 9.5 | 9.5 |
| Extract of Punica Granatum | 0.5 | 0.5 | 0.5 | 0.5 |
| Polylysine | — | — | — | 1.0 |
| pH | 4 | 4 | 4 | 4 |
| Transparency (5 days later) | Very | Good | Good | Very |
| | Good | | | Good |
| Transparency (1 month later) | Good | Good | Good | Very |
| | | | | Good |
| β-Carotene Bleaching Test | Very | Good | Good | Very |
| | Good | | | Good |

TABLE 2

| | Comp. | Comp. | Comp. | Comp. | Comp. | Comp. | Comp. |
|---|---|---|---|---|---|---|---|
| | Ex. 1 | Ex. 2 | Ex. 3 | Ex. 4 | Ex. 5 | Ex. 6 | Ex. 7 |
| Water | qsp | qsp | qsp | qsp | qsp | qsp | qsp |
| | 100 | 100 | 100 | 100 | 100 | 100 | 100 |
| Chitosan | 0.2 | 0.2 | — | — | 0.2 | 0.2 | 0.2 |
| (MW: 100,000) | | | | | | | |
| (Acetylation Degree: 4) | | | | | | | |
| Chitosan | — | — | 0.2 | — | — | — | — |
| (MW: 2,000) | | | | | | | |
| (Acetylation Degree: 3) | | | | | | | |
| Chitosan | — | — | — | 0.2 | — | — | — |
| (MW: 500,000) | | | | | | | |
| (Acetylation Degree: 10) | | | | | | | |
| Ascorbic Acid | 0.1 | 2 | 0.4 | 0.4 | — | — | — |
| Lactic Acid | — | — | — | — | 0.2 | — | — |
| Citric Acid | — | — | — | — | — | 1.5 | — |
| (10 wt % Aqueous Solution) | | | | | | | |
| HCl (1 mol/L) | — | — | — | — | — | — | 1 |
| Dipropylene Glycol | 9.5 | 9.5 | 9.5 | 9.5 | 9.5 | 9.5 | 9.5 |
| Extract of Punica Granatum | 0.5 | 0.5 | 0.5 | 0.5 | 0.5 | 0.5 | 0.5 |
| pH | 5.5 | 2.8 | 4 | 4 | 4 | 4 | 4 |
| Transparency (5 days later) | Poor | Poor | Very | Poor | Very | Very | Very |
| | | | Poor | | Poor | Poor | Poor |
| Transparency (1 month later) | Very | Very | Very | Poor | Very | Very | Very |
| | Poor | Poor | Poor | | Poor | Poor | Poor |
| β-Carotene Bleaching Test | Poor | Poor | Poor | Poor | Very | Very | Very |
| | | | | | Poor | Poor | Poor |

### [Evaluations]

### (Transparency)

Each of the compositions according to Examples 1-4 and the compositions according to Comparative Examples 1-7 was poured into a transparent glass bottle with a volume of 20 ml, and well shaken.

Five days later and one month later, the transparency of each of the compositions according to Examples 1-4 and the compositions according to Comparative Examples 1-7 was evaluated by visual observation in accordance with the following criteria.

Very Good: The composition was transparent, and no particles were found

Good: The composition was transparent, and a very small amount of little particles was found

Poor: A part of the composition was not transparent

Very Poor: The composition was opaque

The results are shown in Tables 1 and 2.

The compositions according to Examples 1-4 were transparent for a long period of time. The composition according to Example 1 including ascorbic acid showed better transparency. The composition according to Example 4 including ascorbic acid and polylysine showed the best transparency.

The transparency indicates that the ingredients, including polyphenol, in the compositions are solubilized.

The compositions according to Comparative Examples 1 and 2 showed poor transparency because they did not satisfy the pH requirements of being 3.0 to 5.0.

The compositions according to Comparative Examples 3 and 4 also showed poor transparency because the molecular weight of chitosan used therein was too low or too high.

The compositions according to Comparative Examples 5-7 also showed poor transparency because they did not use ascorbic acid, gluconolactone, or glycolic acid.

### (β-Carotene Bleaching Test)

0.6 g of a β-carotene solution (solvent: caprylic/capric triglyceride) with a concentration of 0.05% by weight, and 0.2 g of each of the compositions according to Examples 1-4 and the compositions according to Comparative Examples 1-7 were mixed to obtain a mixture.

0.4 g of the above mixture was applied onto a filter (glass microfiber filter).

The above filter was exposed to UV rays with a wavelength of 365 nm for 30 minutes to oxidize β-carotene.

If β-carotene is oxidized, the original yellow color derived from β-carotene fades.

The remaining yellowness of the filter was visually evaluated in accordance with the following criteria.

Very Good: Almost no change

Good: Little change, but acceptable

Poor: Visible change, and not acceptable

Very Poor: Large change

The results are shown in Tables 1 and 2.

The stronger the remaining yellowness was, the more β-carotene remained. This means that the stronger the remaining yellowness was, the more the anti-oxidation effect by the polyphenol in the composition was.

The compositions according to Examples 1-4 exhibited good anti-oxidation effects. The composition according to Example 1 including ascorbic acid and the composition according to Example 4 including ascorbic acid and polylysine exhibited the best anti-oxidative effect.

The compositions according to Comparative Examples 1 and 2 exhibited poor anti-oxidative effect because they did not satisfy the pH requirements of being 3.0 to 5.0.

The compositions according to Comparative Examples 3 and 4 also exhibited poor anti-oxidative effect because the molecular weight of chitosan used therein was too low or too high.

The compositions according to Comparative Examples 5-7 also exhibited poor anti-oxidative effect because they did not use ascorbic acid, gluconolactone, or glycolic acid.

1. A composition, comprising:
(a) at least one polyphenol;
(b) at least one first cationic polymer with a molecular weight of from 5,000 to 450,000 selected from chitosans;
(c) at least one organic compound selected from the group consisting of ascorbic acid, gluconolactone, gluconic acid, glycolic acid, and mixtures thereof; and
(d) water
wherein
the pH of the composition is from 3.0 to 5.0.

2. The composition according to claim 1, wherein the (a) polyphenol is selected from ellagic acid, punicalagins, and mixtures thereof.

3. The composition according to claim 1, wherein the (a) polyphenol is derived from plants.

4. The composition according to claim 1, wherein the amount of the (a) polyphenol(s) in the composition is from 0.001% to 10% by weight relative to the total weight of the composition.

5. The composition according to claim 1, wherein the (b) first cationic polymer is selected from chitosans with an acetylation degree of more than 3% and less than 10%.

6. The composition according to claim 1, wherein the amount of the (b) first cationic polymer(s) in the composition is from 0.001% to 10% by weight relative to the total weight of the composition.

7. The composition according to claim 1, wherein the amount of the (c) organic compound(s) in the composition is from 0.001% to 15% by weight, relative to the total weight of the composition.

8. The composition according to claim 1, wherein the amount of the (d) water in the composition is from 50% to 98% by weight relative to the total weight of the composition.

9. The composition according to claim 1, wherein the composition further comprises (e) at least one second cationic polymer selected from polylysines.

10. The composition according to claim 10, wherein the amount of the (e) second cationic polymer(s) in the composition is from 0.001% to 10% by weight relative to the total weight of the composition.

11. The composition according to claim 1, wherein the composition further comprises (f) at least one polyol.

12. The composition according to claim 11, wherein the (f) polyol is dipropyleneglycol.

13. The composition according to claim 11, wherein the amount of the (f) polyol(s) in the composition is from 0.1% to 25% by weight relative to the total weight of the composition.

14. The composition according to claim 1, wherein the composition is a cosmetic composition.

15. A cosmetic process for a keratin substance such as skin, comprising:
applying to the keratin substance the composition according to claim 1.
